(19) United States
(12) Patent Application Publication
(10) Pub. No.: US 2024/0350756 A1
(43) Pub. Date: Oct. 24, 2024

(54) DETERMINATION OF A GAS CONCENTRATION IN A VENTILATION SYSTEM

(71) Applicant: Loewenstein Medical Technology S.A., Luxembourg (LU)

(72) Inventor: Petrus Johannes Josephus BORM, HP Eindhoven (NL)

(21) Appl. No.: 18/624,291

(22) Filed: Apr. 2, 2024

(30) Foreign Application Priority Data
Apr. 18, 2023 (DE) 102023109772.5

(51) Int. Cl. A61M 16/00

(52) U.S. Cl. CPC A61M 16/024; A61M 16/0003; A61M 2206/10

(57) ABSTRACT

A method for determining a gas concentration in a ventilation system comprises receiving a first signal, which indicates a gas concentration measured by a gas sensor at at least one first point in a secondary line, in a signal processing device; generating a second signal, which indicates an estimated gas concentration at at least one second point, differing from the first point, in a main line, from the first signal by the signal processing device. The main line connects a fitting of a ventilator for providing a respiratory gas to a patient interface and the secondary line branches off from the main line so that the respiratory gas can flow at least partially through the secondary line.

### CROSS-REFERENCE TO RELATED APPLICATIONS

The present application claims priority under 35 U.S.C. § 119 of German Patent Application No. 10 2023 109 772.5, filed Apr. 18, 2023, the entire disclosure of which is expressly incorporated by reference herein.

### BACKGROUND OF THE INVENTION

### 1. Field of the Invention

The invention relates to a method for determining a gas concentration in a ventilation system. Furthermore, the invention relates to a signal processing device, a computer program, and a computer-readable medium for carrying out the method, as well as a ventilation system.

### 2. Discussion of Background Information

The concentration of carbon dioxide in a respiratory gas, which flows in a ventilation system, can be determined by a main flow measurement or a secondary flow measurement. In the main flow measurement, the concentration is directly measured via a gas sensor arranged in the airway, i.e., in the main flow of the respiratory gas. Such a measurement is generally very accurate. However, the gas sensor requires a certain minimum volume for accurate measurement results, which in particular in the ventilation of newborns or infants can result in undesired rebreathing of carbon dioxide. Such a gas sensor can also be unpleasant for the patient due to its size and its weight. In the secondary flow measurement, a part of the respiratory gas is branched off at the respective removal point, for example, a nasal cannula or a mask, and led to an external gas sensor, which can be arranged in the ventilator. Such a secondary flow measurement is generally less accurate than the main flow measurement.

In view of the foregoing it would be advantageous to have available a method which enables the most accurate possible determination of a gas concentration in a ventilation system and avoids the above-mentioned disadvantages of a conventional main flow measurement. It further would be advantageous to have available a signal processing device, a computer program, and a computer-readable medium for carrying out such a method, as well as a corresponding ventilation system.

### SUMMARY OF THE INVENTION

The present invention provides a method, a signal processing device, a computer program, and a computer-readable medium for carrying out the method, as well as a corresponding ventilation system as set forth in the claims.

A first aspect of the invention relates to a method for determining a gas concentration in a ventilation system. The ventilation system comprises: a ventilator having a fitting for providing a respiratory gas; a main line for connecting the fitting to a patient interface; a secondary line, which branches off from the main line, so that the respiratory gas can flow at least partially through the secondary line; a gas sensor for measuring a gas concentration at at least one first point in the secondary line; a signal processing device. The method comprises the following steps: receiving a first signal, which indicates the gas concentration measured by the gas sensor, in the signal processing device; generating a second signal, which indicates an estimated gas concentration at at least one second point, differing from the first point, in the main line, from the first signal by way of the signal processing device.

In other words, the gas concentration can be directly measured at a specific point in the secondary line, i.e., in the secondary flow. The gas concentration can then be estimated from the resulting sensor signal at another point in the main line, i.e., in the main flow, for example, using a mathematical model of the gas sensor and a known volume flow of the respiratory gas in the main line and/or the secondary line (see below).

Using such an estimation, a similar accuracy can be achieved as with a conventional main flow measurement, without the gas sensor having to be integrated into the main line. The total volume of the main line is thus reduced. This can reduce the probability of undesired rebreathing of exhaled respiratory gas. Moreover, the total weight of the main line is reduced. This can improve the comfort for the patient, in particular in the ventilation of newborns or infants.

The method can be computer-implemented.

A “signal” as in “first signal”, “second signal”, or “third signal” (see below) can be understood as an analog or digital signal.

A second aspect of the invention relates to a signal processing device which comprises means for carrying out the method described above and below.

The means can comprise hardware and/or software modules. In particular, the means can comprise a processor which is configured to carry out the (computer-implemented) method. In addition, the means can comprise a memory and/or a data communication interface for wireless and/or wired data communication with peripheral devices. Alternatively, the signal processing device can exclusively be implemented as hardware, for example, in the form of an ASIC or FPGA module.

It is to be noted that features of the method described above and below can also be features of the signal processing device (and vice versa).

A third aspect of the invention relates to a ventilation system. The ventilation system comprises: a ventilator having a fitting for providing a respiratory gas; a main line for connecting the fitting to a patient interface; a secondary line, which branches off from the main line, so that the respiratory gas can flow at least partially through the secondary line; a gas sensor for measuring a gas concentration at at least one first point in the secondary line; a signal processing device as described above and below.

A “ventilator” can be understood, for example, as a home ventilator, a sleep respiratory therapy device, an intensive ventilator, an anesthesia device, or a combination of at least two of these examples. The ventilator can be designed for invasive and/or noninvasive ventilation.

A “line” as in “main line” or “secondary line” can be understood, for example, as a (flexible) tube. However, rigid lines are also possible.

The respiratory gas flowing through the main line can also be designated as the main flow. The respiratory gas flowing through the secondary line can also be designated as the secondary flow.

A “patient interface” can be understood, for example, as an intubation tube, a nasal cannula, or a breathing mask.

The secondary line can, for example, differ significantly from the main line in its total volume and/or its total length.

A “gas sensor” can be understood, for example, as an infrared-optical sensor, also called a non-dispersive infrared sensor or NDIR sensor in short, or a metal oxide semiconductor sensor, called MOX sensor in short. A gas sensor is also possible which measures the gas concentration on the basis of the thermal conductivity of the respective gas or gas mixture, also called a thermal conductivity detector, or paramagnetically.

The gas sensor can be arranged at least partially, for example, with its active surface, in the secondary line, in order to measure the gas concentration directly in the secondary line.

Further aspects of the invention relate to a computer program and a computer-readable medium on which the computer program is stored.

The computer program comprises commands which cause a processor (for example, a processor of the signal processing device described above and below) upon execution of the computer program by the processor to carry out the method described above and below.

The computer-readable medium can be a volatile or nonvolatile data memory. For example, the computer-readable medium can be a hard drive, a USB storage device (universal serial bus), a RAM (random-access memory), a ROM (read-only memory), an EPROM (erasable programmable read-only memory), an EEPROM (electrically erasable programmable read-only memory), a flash memory, or a combination of at least two of these examples. The computer-readable medium can also be a data communication network, which enables the downloading of program code (for example via the Internet), or a cloud.

It is to be noted that features of the method described above and below can also be features of the computer program and/or the computer-readable medium (and vice versa).

Various embodiments of the invention are described hereinafter. These embodiments are not to be understood as a restriction of the scope of the invention.

According to one embodiment, the estimated gas concentration can be determined depending on a time derivative of the measured gas concentration. In other words, at least a part of the second signal can correspond to a time derivative of the first signal or can be based thereon. In this way, the gas concentration at the second point or the second points can be estimated sufficiently accurately. Such an estimation moreover has the advantage that it can be implemented very easily as hardware and/or software.

According to one embodiment, a relationship between the measured gas concentration and the estimated gas concentration can be defined by the following equation:

$$\frac{d}{dt} ( ConcSensor ) = \frac{1}{τ} ( ConcAirway - ConcSensor ) .$$

In this case, “ConcSensor” stands for the measured gas concentration, “ConcAirway” stands for the estimated gas concentration, and t stands for a constant, for example, a time constant.

In other words, it is possible that the second signal is generated using from the first signal a first-order (reverse) filter.

The equation can be transformed and/or discretized, i.e., digitized, for example, by Laplace transformation in order to generate the second signal, i.e., to calculate values for the estimated gas concentration. The equation can be stored, for example, as a mathematical function or lookup table in a memory of the signal processing device.

According to one embodiment, the at least one second point can comprise a branching point, at which the secondary line branches off from the main line. The branching point can be, for example, a point at which the gas concentration would be measured (instead of estimated as here) in a conventional main flow measurement. In other words, the first signal can be evaluated to estimate the gas concentration at least at the branching point. This enables, for example, a very accurate determination of the amount of exhaled carbon dioxide.

According to one embodiment, the second signal can be generated using a mathematical model of the gas sensor. The mathematical model can comprise, for example, a system of differential equations for defining a relationship between the first and the second signal. The mathematical model can be defined by specific parameters which describe physical and/or chemical properties of the gas sensor. The mathematical model can be used to approximately simulate a signal of the gas sensor which the gas sensor would generate if it were to measure the gas concentration, instead of at the first point or the first points, at the second point or the second points. The mathematical model can be defined, for example, by at least one of the following parameters: one or more time constants, a volume of the main line and/or the secondary line, a volume flow of the respiratory gas in the main line and/or the secondary line, a dead volume. The time constant can be, for example, a quotient of the volume and the volume flow. The mathematical model can have been determined, for example, by analysis of a jump response, which results from a sudden change of the gas concentration at the second point or the second points.

According to one embodiment, the second signal can be generated depending on a known measurement volume of the gas sensor. “Measurement volume” can be understood as the volume of a measurement chamber of the gas sensor through which the respiratory gas flows. The known measurement volume can be, for example, a parameter of the mathematical model of the gas sensor.

According to one embodiment, the second signal can be generated depending on a known volume flow of the respiratory gas in the main line.

According to one embodiment, the second signal can be generated depending on a known volume flow of the respiratory gas in the secondary line.

A known volume flow, also called a flow, can be, for example, a measured volume flow and/or can be predetermined by the known properties of the main line and/or the secondary line. The known volume flow of the respiratory gas in the main line and/or the secondary line can be, for example, a parameter of the mathematical model of the gas sensor.

According to one embodiment, the known volume flow of the respiratory gas in the secondary line, independently of a (known or unknown) pressure (and/or volume flow) of the respiratory gas in the main line, can either have a constant absolute value or a constant direction or both a constant absolute value and a constant direction. In other words, the volume flow in the secondary line can be controlled so that it remains more or less equal, even if the pressure (and/or the volume flow) significantly changes in the main line or in a partial section of the main line, for example, at the transition between inhalation and exhalation.

According to one embodiment, the known volume flow of the respiratory gas in the secondary line can be 2 mL/s or less, in particular 1 mL/s or less. Such values are particularly favorable for the ventilation of newborns or infants.

According to one embodiment, τ=VolumeSensor/Flow can apply. In this case, “VolumeSensor” stands for the known measurement volume of the gas sensor and “Flow” stands for the known volume flow of the respiratory gas in the secondary line. In other words, the relationship between the measured gas concentration and estimated gas concentration can be defined by the following equation:

$$\frac{d}{dt} ( ConcSensor ) = Flow / VolumeSensor * ( ConcAirway - ConcSensor ) .$$

According to one embodiment, the measured gas concentration can be a carbon dioxide concentration.

According to one embodiment, the estimated gas concentration can be a carbon dioxide concentration.

According to one embodiment, the method can furthermore comprise a step of filtering the second signal to smooth the second signal. Examples of suitable filters are bandpass, high-pass, or low-pass filters. However, other filters are also possible. In this way, for example, undesired noise in the second signal can be suppressed. The accuracy in the determination of the gas concentration can thus be further improved.

According to one embodiment, the second signal can be filtered using a low-pass filter. Undesired noise in the second signal can thus be suppressed particularly effectively using simple means.

According to one embodiment, a third signal, which indicates a pressure difference measured by a pressure sensor between various points of the main line, can also be received in the signal processing device. The second signal can then be generated while additionally using the third signal. The third signal can be used, for example, to determine a volume flow of the respiratory gas in the main line and/or the secondary line and/or a ventilation pressure and/or to activate a pump of the ventilation system (see below) so that a volume flow of the respiratory gas in the main line and/or the secondary line approaches a specific target value. With respect to the volume flow of the respiratory gas in the secondary line, the target value can be, for example, 2 mL/s or less, in particular 1 mL/s or less.

According to one embodiment, the ventilation system can furthermore comprise a respiratory gas filter for filtering the respiratory gas before it enters the gas sensor. Measurement errors as a result of contaminants and/or damage of the gas sensor can thus be avoided.

According to one embodiment, the respiratory gas filter can comprise either a water trap or a particle filter or both a water trap and a particle filter. A “particle filter” can be understood above and below in particular as a hydrophobic particle filter, which can additionally remove moisture from the respiratory gas.

According to one embodiment, the ventilation system, in particular the secondary line, can be designed so that a volume flow of the respiratory gas in the secondary line, independently of a pressure (and/or a volume flow) of the respiratory gas in the main line, either has a constant absolute value or a constant direction or both a constant absolute value and a constant direction. It can thus be ensured that the measurement of the gas concentration takes place at various points in time under approximately identical measurement conditions.

According to one embodiment, the ventilation system, in particular the secondary line, can be designed so that a volume flow of the respiratory gas in the secondary line is 2 mL/s or less, in particular 1 mL/s or less. Such values are particularly favorable for the ventilation of newborns or infants.

According to one embodiment, the secondary line can comprise an aperture for setting the volume flow of the respiratory gas in the secondary line. An “aperture” can be understood as a specially shaped, in particular sudden local constriction of the cross section of the secondary line, for example, in the form of a disk or a perforated grid, wherein the local constriction forms a flow resistance. The aperture can have, for example, an opening having a diameter varying in the flow direction. It was possible to achieve very good results in experiments using such an aperture. Alternatively, a throttle or a control valve can also be used.

According to one embodiment, the secondary line can comprise a first line section and a second line section. In this case, the gas sensor can be arranged in the first line section and the second line section can bypass the gas sensor. Such a bypass enables the volume flow of the respiratory gas through the secondary line to be maintained even if the path via the gas sensor is blocked or constricted for any reason.

According to one embodiment, the first line section can comprise a first aperture for setting a volume flow of the respiratory gas in the first line section. In this way, the volume flow in the first line section can be set to a specific value with little design effort.

According to one embodiment, the second line section can comprise a second aperture for setting a volume flow of the respiratory gas in the second line section. In this way, the volume flow in the second line section can be set to a specific value with little design effort.

It can be ensured by the combination of the two apertures that the volume flow of the respiratory gas—in particular where it enters the gas sensor—approaches a specific target value. For example, the second aperture can have a smaller opening than the first aperture. This has the effect that a sufficiently strong volume flow is generated in the second line section if the first line section is blocked for any reason, in particular if a respiratory gas filter upstream of the gas sensor in the first line section (also called the first respiratory gas filter below) is clogged, so that the clogged respiratory gas filter can accordingly be flushed and made passable again.

According to one embodiment, the first aperture can be arranged between an outlet opening of the gas sensor and an orifice point, at which the second line section opens into the first line section.

According to one embodiment, the first line section can comprise a first respiratory gas filter. The first respiratory gas filter can comprise, for example, a particle filter and/or a water trap. The first respiratory gas filter can be arranged so that it is flushed by the volume flow of the respiratory gas in the second line section. In this way, for example, mucus or moisture can be removed from the first respiratory gas filter via the second line section. Clogs of the first respiratory gas filter can thus be avoided or eliminated.

According to one embodiment, the second line section can comprise a second respiratory gas filter. The second respiratory gas filter can comprise, for example, a particle filter and/or a water trap.

The first and the second respiratory gas filter can be of the same type or of different types.

According to one embodiment, an outlet of the first respiratory gas filter can be connected to an entry opening of the gas sensor. Additionally or alternatively, the outlet can be connected to an inlet of the second respiratory gas filter. In other words, the first respiratory gas filter can be connected to the gas sensor so that the gas sensor can measure the gas concentration in the respiratory gas filtered by the first respiratory gas filter. The first and the second respiratory gas filter can be connected in series to one another and/or to at least one third respiratory gas filter in order to enable a two-stage or multistage filtering of the respiratory gas.

According to one embodiment, the outlet of the first respiratory gas filter can be connected via at least one third respiratory gas filter to the inlet of the second respiratory gas filter. In other words, at least three respiratory gas filters can be connected to one another in series in order to filter the respiratory gas. Each respiratory gas filter can comprise, for example, a particle filter and/or a water trap.

According to one embodiment, the first respiratory gas filter can comprise a first particle filter, the second respiratory gas filter can comprise a second particle filter, and the at least one third respiratory gas filter can comprise a water trap. This enables particularly thorough filtering of the respiratory gas.

According to one embodiment, the ventilation system can comprise a pump arranged in the main line or the secondary line for conveying the respiratory gas. Alternatively, at least one first pump can be arranged in the main line and at least one second pump can be arranged in the secondary line. The pump can instead also be arranged in the ventilator. The pump can be designed, for example, to generate a negative pressure in the main line and/or the secondary line. For example, the negative pressure can deviate from ambient pressure by a factor of from about 0.4 to about 0.6, in particular from about 0.50 to about 0.55. It is possible that the pump is activated using the first signal, the second signal, or the third signal or using at least two of these signals. For example, a blockage or another undesired cross-sectional constriction of the main line can be detected on the basis of the third signal. As a reaction thereto, the pump can be activated so that negative pressure does not arise in the alveoli of the ventilated patient, in particular a newborn or infant. For example, the pump can be deactivated during the ventilation of a newborn or infant if the measured pressure in the main line exceeds a specific threshold, for example, falls under 2 mbar.

Additionally or alternatively, a current (positive or negative) value of a ventilation pressure can be determined from the third signal, using which, for example, the pump can be activated so that the current value approaches a specific target value.

According to one embodiment, the pump can be arranged in the first line section and the second line section can open into the first line section between the pump and the gas sensor.

According to one embodiment, the ventilation system can comprise a suction device arranged in the main line or the secondary line for aspirating liquid, in particular secretion or mucus. Clogs can thus be avoided.

According to one embodiment, the suction device can be arranged in the main line between a branching point, at which the secondary line branches off from the main line, and the patient interface. In this way, it is possible to prevent liquid, in particular secretion or mucus, from penetrating into the main line and the secondary line upon exhalation.

Alternatively, the suction device can be arranged in the main line between the fitting of the ventilator and a branching point, at which the secondary line branches off from the main line. For example, the suction device can be arranged here between a point at which a pressure sensor is connected to the main line and the branching point.

According to one embodiment, the ventilation system can comprise a pressure sensor for measuring a pressure difference between various points of the main line. The pressure sensor can be connected, for example, to the signal processing device for data communication.

According to one embodiment, the ventilation system can comprise an additional pressure sensor for measuring a ventilation pressure, using which the patient is currently ventilated, for example, at the branching point or at another point as close as possible to the patient interface.

According to one embodiment, a first pressure fitting of the pressure sensor can be connected to the main line between the fitting of the ventilator and a branching point, at which the secondary line branches off from the main line. A second pressure fitting of the pressure sensor can be connected to the main line between a point at which the first pressure fitting is connected to the main line and the patient interface.

According to one embodiment, the second pressure fitting can be connected to the main line between the fitting of the ventilator and the branching point.

According to one embodiment, the second pressure fitting can be connected to the main line at the branching point.

According to one embodiment, the secondary line can have an opening out of which the respiratory gas can flow into the surroundings. For example, the opening can be formed by a free open end of the secondary line, so that the respiratory gas can flow into the surroundings after it has passed the gas sensor.

According to one embodiment, the ventilator can comprise a further fitting for connecting the secondary line, so that the respiratory gas can flow out of the secondary line back into the ventilator. More precisely, the respiratory gas, after it has passed the gas sensor, can flow via the further fitting back into the ventilator again. The ventilator can thus provide the respiratory gas which has flowed back, for example, after corresponding preparation in the ventilator, at the fitting connected to the main line again. This can improve the efficiency of the ventilation system.

According to one embodiment, the ventilation system can be designed for ventilating newborns and/or infants.

### BRIEF DESCRIPTION OF THE DRAWINGS

Embodiments of the invention are described hereinafter with reference to the appended drawings. Neither the description nor the drawings are to be understood as a restriction of the scope of the invention. In the drawings,

FIG. 1 shows a ventilation system according to one embodiment of the invention; and

FIG. 2 shows a diagram to illustrate a signal as is generated in a method according to one embodiment of the invention.

The drawings are solely schematic and are not to scale. If identical reference signs are used in different drawings, these reference signs designate identical or identically acting features.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

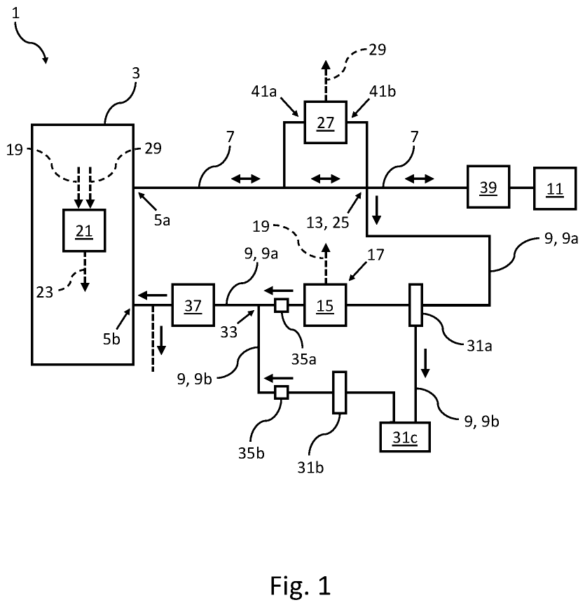

FIG. 1 shows a ventilation system 1, which comprises a ventilator 3 having a fitting 5a for providing a respiratory gas, a main line 7, and a secondary line 9. The main line 7 connects the fitting 5a to a patient interface 11, for example, an intubation tube, a nasal cannula, or a breathing mask. The secondary line 9 branches off at a branching point 13 from the main line 7, so that the respiratory gas can flow during the ventilation not only between the fitting 5a and the patient interface 11, but also partially through the secondary line 9. Possible flow directions of the respiratory gas are marked by continuous arrows in FIG. 1.

The ventilation system 1 furthermore comprises a gas sensor 15 for measuring a gas concentration at at least one first point 17 in the secondary line 9. The gas sensor 15, for example, a carbon dioxide sensor, can be arranged at least partially in the secondary line 9. The gas sensor 15 is designed to generate a first signal 19, which indicates the gas concentration measured at the first point 17 or the first points 17. The gas sensor 15 is connected to a signal processing device 21 of the ventilation system 1 for data communication. The signal processing device 21 can be a component of the ventilator 3, as shown here.

The signal processing device 21 is designed to carry out a method for determining a gas concentration in the ventilation system 1. For this purpose, the signal processing device 21 can comprise, for example, a memory and a processor (not shown). A computer program can be stored in the memory and the processor can be configured to carry out the method by executing the computer program.

The method comprises a first step, in which the first signal 19 is received in the signal processing device 21 via a suitable data communication connection, which can be wired or wireless. In a second step, the signal processing device 21 generates a second signal 23 from the first signal 19, which indicates an estimated gas concentration at at least one second point 25, differing from the first point 17, in the main line 7. In this example, the second point 25 corresponds to the branching point 13. However, the second point 25 can also be a different point of the main line 7.

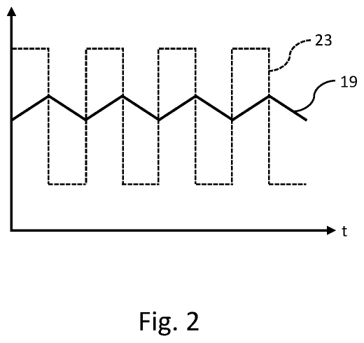

In the simplest case, the signal processing device 21 generates the second signal 23 depending on a time derivative of the first signal 19, as shown by way of example in FIG. 2. The signals 19, 23 are shown in idealized form in FIG. 2. In reality, the signals 19, 23 can have a slightly differing form, for example, can be more or less noisy.

Additionally or alternatively, the second signal 23 can be generated using a mathematical model of the gas sensor 15.

The estimated gas concentration can be determined particularly accurately in that the first signal 19, i.e., the measured gas concentration, is processed according to the following equation:

$$\frac{d}{dt} ( ConcSensor ) = Flow / VolumeSensor * ( ConcAirway - ConcSensor ) .$$

In this case, “ConcSensor” stands for the gas concentration measured at the first point 17 or the first points 17, “Flow” stands for a known volume flow of the respiratory gas in the secondary line 9, “VolumeSensor” stands for a known measurement volume of the gas sensor 15, and “ConcAirway” stands for the estimated gas concentration.

The ventilation system 1, in particular the main line 7 and/or the secondary line 9, can be designed so that the volume flow in the secondary line 9 is approximately constant, i.e., has the same absolute value in the same direction, even if the pressure in the main line 7 changes, for example, during the transition between inhalation and exhalation. In particular in the ventilation of newborns or infants, it is favorable if the volume flow in the secondary line 9 is 2 mL/s or less or even 1 mL/s or less.

In addition, the second signal 23 can be smoothed by the signal processing device 21, for example, using a low-pass filter, in order to suppress undesired noise.

It is also possible that in the estimation of the gas concentration at the second point 25 or the second points 25, a third signal 29 provided by a pressure sensor 27 is taken into consideration, wherein the third signal 29 indicates a pressure difference between various points of the main line 7. This can further improve the accuracy of the estimation. For example, at least one of the following variables can be determined on the basis of the (measured) pressure difference: a current volume flow between the fitting 5a and the branching point 13, a current volume flow between the branching point 13 and the patient interface 11, a current volume flow in the secondary line 7, a current ventilation pressure in the main line 7, in particular at the branching point 13.

With the aid of the above-described method, the gas concentration, the carbon dioxide concentration here, can be determined at one or more specific points in the main line 7 in real time without a corresponding gas sensor having to be placed in the main line 7, i.e., in the main flow. This has the advantage in particular in the ventilation of newborns and/or infants that, due to the reduced volume of the main line 7, no or significantly less exhaled respiratory gas is inhaled again. In addition, the main line 7 is lighter due to the arrangement of the gas sensor 15 in the secondary line 9, which simplifies the handling and improves the comfort for the patient.

In the example shown in FIG. 1, the secondary line 9 comprises a first line section 9a and a second line section 9b, wherein the gas sensor 15 is arranged in the first line section 9a and the second line section 9b bypasses the gas sensor 15 arranged in the first line section 9a.

A first respiratory gas filter 31a, for example, in the form of a first particle filter, can be arranged in the first line section 9a between the branching point 13 and an entry opening of the gas sensor 15, through which the respiratory gas enters the gas sensor 15, so that the respiratory gas is filtered before it enters the gas sensor 15.

Additionally or alternatively, a second respiratory gas filter 31b, for example, in the form of a second particle filter, can be arranged in the second line section 9b, so that the respiratory gas is filtered before it leaves the second line section 9b.

It is possible that at least one third respiratory gas filter 31c is arranged between the respiratory gas filters 31a, 31b, for example, in the form of a water trap in the second line section 9b. This has the effect that the respiratory gas exiting from the first respiratory gas filter 31a is additionally filtered before it enters the second respiratory gas filter 31b.

To keep the volume flow in the secondary line 9 constant, the secondary line 9 can have one or more apertures.

In this example, a first aperture 35a, which sets the volume flow in the first line section 9a, is arranged between an outlet opening of the gas sensor 15, through which the respiratory gas exits from the gas sensor 15, and an orifice point 33, at which the second line section 9b opens into the first line section 9a.

In addition, a second aperture 35b, which sets the volume flow in the second line section 9b, can be arranged in the second line section 9b, for example, between the orifice point 33 and an outlet of the second respiratory gas filter 31b.

More precisely, the first aperture 35a only sets a part of the volume flow in the first line section 9a in this case. The other part is set by the second aperture 35b. The (total) volume flow through the first line section 9a therefore corresponds to a sum of the volume flow through the first aperture 35a and the volume flow through the second aperture 35b.

The apertures 35a, 35b can be designed so that the total volume flow in the secondary line 9, in particular the volume flow between the branching point 13 and the entry opening of the gas sensor 15, is 2 mL/s or less or even 1 mL/s or less.

To convey the respiratory gas, a suitable pump 37 can be arranged in the secondary line 9, for example in the first line section 9a between the orifice point 33 and an end of the secondary line 9. The pump 37 can alternatively be arranged in the main line 7 or in the ventilator 3.

The end of the secondary line 9 can be open, so that the respiratory gas can flow into the surroundings, as indicated by a dashed line in FIG. 1.

Alternatively, the end of the secondary line 9 can be connected to a further fitting 5b of the ventilator 3, so that the respiratory gas can flow out of the secondary line 9 back into the ventilator 3, in order to be conducted from there, for example, after a suitable preparation in the ventilator 3, back into the main line 7.

To avoid clogs or other negative effects in the ventilation as a result of secretions or condensed water, the ventilation system 1 can comprise a suitable suction device 39 for aspirating and collecting such liquids. In this example, the suction device 39 is arranged in the main line 7 between the branching point 13 and the patient interface 11. In this way, inter alia, clogs of the pressure sensor 27 can be avoided.

It is possible that a first pressure fitting 41a of the pressure sensor 27 is connected to the main line 7 between the suction device 39 and the branching point 13 and a second pressure fitting 41b of the pressure sensor 27 is connected to the main line 7 between the branching point 13 and the patient interface 11. A configuration is also possible in which both pressure fittings 41a, 41b are connected to the main line 7 between the fitting 5a and the branching point 13.

Finally, it is to be noted that terms such as “having”, “comprising”, “including”, “with”, etc. do not exclude other elements or steps and indefinite articles such as “a” or “an” do not exclude multiples.

Furthermore, it is to be noted that features or steps which are described with reference to one of the above embodiments can also be used in combination with features or steps which are described with reference to other ones of the above embodiments.

### LIST OF REFERENCE SIGNS

1 ventilation system
3 ventilator
5a fitting
5b further fitting
7 main line
9 secondary line
9a first line section
9b second line section
11 patient interface
13 branching point
15 gas sensor
17 first point
19 first signal
21 signal processing device
23 second signal
25 second point
27 pressure sensor
29 third signal
31a first respiratory gas filter
31b second respiratory gas filter
31c third respiratory gas filter
33 orifice point
35a first aperture
35b second aperture
37 pump
39 suction device
41a first pressure fitting
41b second pressure fitting

What is claimed is:

1. A method for determining a gas concentration in a ventilation system which comprises
a ventilator having a fitting for providing a respiratory gas;
a main line for connecting the fitting to a patient interface;
a secondary line, which branches off from the main line, so that the respiratory gas can flow at least partially through the secondary line;
a gas sensor for measuring a gas concentration at at least one first point in the secondary line; and
a signal processing device;
wherein the method comprises:
receiving a first signal, which indicates the gas concentration measured by the gas sensor, in the signal processing device;
generating a second signal, which indicates an estimated gas concentration at at least one second point, differing from the first point, in the main line, from the first signal by the signal processing device.

2. The method of claim 1, wherein the estimated gas concentration is determined depending on a time derivative of the measured gas concentration.

3. The method of claim 1, wherein a relationship between the measured gas concentration and the estimated gas concentration is defined by the following equation:
$$\frac{d}{dt} ( ConcSensor ) = \frac{1}{τ} ( ConcAirway - ConcSensor ) ,$$
wherein “ConcSensor” represents a measured gas concentration, “ConcAirway” represents an estimated gas concentration, and τ is a constant.

4. The method of claim 1, wherein the at least one second point comprises a branching point at which the secondary line branches off from the main line.

5. The method of claim 1, wherein the second signal is generated using a mathematical model of the gas sensor and/or wherein the second signal is generated depending on a known measurement volume of the gas sensor and/or depending on a known volume flow of the respiratory gas in the main line and/or the secondary line.

6. The method of claim 5, wherein the known volume flow of the respiratory gas in the secondary line, independent of a pressure of the respiratory gas in the main line, has a constant absolute value and/or a constant direction.

7. The method of claim 6, wherein the constant absolute value is 2 mL/s or less.

8. The method of claim 6, wherein the constant absolute value is 1 mL/s or less.

9. The method of claim 5, wherein a relationship between the measured gas concentration and the estimated gas concentration is defined by the following equation:
$$\frac{d}{dt} ( ConcSensor ) = \frac{1}{τ} ( ConcAirway - ConcSensor ) ,$$
wherein “ConcSensor” represents a measured gas concentration, “ConcAirway” represents an estimated gas concentration, and τ=VolumeSensor/Flow, wherein “VolumeSensor” represents a known measurement volume of the gas sensor and “Flow” represents a known volume flow of the respiratory gas in the secondary line.

10. The method of claim 1, wherein furthermore a third signal, which indicates a pressure difference measured by a pressure sensor between various points of the main line is received in the signal processing device and wherein the second signal is generated using the third signal.

11. A signal processing device, wherein the device is configured to be suitable for carrying out the method of claim 1.

12. A ventilation system, wherein the system comprises:
a ventilator having a fitting for providing a respiratory gas;
a main line for connecting the fitting to a patient interface;
a secondary line, which branches off from the main line, so that the respiratory gas can flow at least partially through the secondary line;
a gas sensor for measuring a gas concentration at at least one first point in the secondary line; and
the signal processing device of claim 11.

13. The ventilation system of claim 12, wherein the ventilation system is configured so that a volume flow of the respiratory gas in the secondary line, independent of a pressure of the respiratory gas in the main line, has a constant absolute value and/or a constant direction.

14. The ventilation system of claim 13, wherein the constant absolute value is 2 mL/s or less.

15. The ventilation system of claim 13, wherein the constant absolute value is 1 mL/s or less.

16. The ventilation system of claim 12, wherein the secondary line comprises a first line section and a second line section, the gas sensor being arranged in the first line section and the second line section bypassing the gas sensor.

17. The ventilation system of claim 16, wherein the first line section comprises a first aperture for setting a volume flow of the respiratory gas in the first line section and/or a first respiratory gas filter; and/or wherein the second line section comprises a second aperture for setting a volume flow of the respiratory gas in the second line section and/or a second respiratory gas filter.

18. The ventilation system of claim 17, wherein the first line section comprises a first aperture for setting a volume flow of the respiratory gas in the first line section and/or a first respiratory gas filter; and the second line section comprises a second aperture for setting a volume flow of the respiratory gas in the second line section and/or a second respiratory gas filter.

19. A computer program, wherein the program comprises commands which cause a processor upon execution of the computer program by the processor to carry out the method of claim 1.

20. A computer-readable medium on which the computer program of claim 19 is stored.
